(19) United States
(12) Patent Application Publication
(10) Pub. No.: US 2026/0223589 A1
(43) Pub. Date: Jul. 30, 2026

(54) COMPOSITE MATERIAL, PREPARATION METHOD THEREOF AND PHOTOELECTRIC DEVICE

(71) Applicant: TCL Technology Group Corporation, Huizhou, Guangdong (CN)

(72) Inventor: Siyu XIA, Huizhou, Guangdong (CN)

(73) Assignee: TCL Technology Group Corporation, Huizhou, Guangdong (CN)

(21) Appl. No.: 19/144,455

(22) PCT Filed: Oct. 27, 2023
(86) PCT No.: PCT/CN2023/127318

§ 371 Date: Jun. 27, 2025

(30) Foreign Application Priority Data
Dec. 28, 2022 (CN) 202211702327.4

(51) Int. Cl. H10K 85/30; B82Y 30/00; B82Y 40/00; C09C 1/04; C09C 1/36; C09C 3/08; H10K 50/16; H10K 50/17

(52) U.S. Cl. CPC H10K 85/381; C09C 1/043; C09C 1/3669; C09C 3/08; H10K 85/341; B82Y 30/00; B82Y 40/00; C01P 2002/54; C01P 2004/64; C01P 2006/40; H10K 50/16; H10K 50/171

(57) ABSTRACT

A composite material, a preparation method thereof and a photoelectric device are disclosed, and related to a field of display technologies. The composite material includes an inorganic particle and a perfluorosulfonamide, wherein the inorganic particle is connected to the perfluorosulfonamide by a chemical bond, and a chemical formula of the perfluorosulfonamide is R—SO₂—NH₂. Perfluorosulfonamide in the composite material could reduce the surface defects of the inorganic particle, inhibit the growth and agglomeration of the inorganic particle, and thus improve the storage time of the inorganic particle.

The present disclosure claims priority to Chinese Present disclosure NO. 202211702327.4 filed in the China National Intellectual Property Administration on Dec. 28, 2022 and entitled “COMPOSITE MATERIAL, PREPARATION METHOD THEREOF, PHOTOELECTRIC DEVICE AND DISPLAY DEVICE”, the content of which is incorporated herein by reference in its entirety.

### TECHNICAL FIELD

The present disclosure relates to a field of display technologies, and more particularly, to composite material, preparation method thereof and photoelectric device.

### BACKGROUND

At present, inorganic semiconductor particles are usually prepared by a low-temperature method or a solution method. The inorganic semiconductor particles prepared by these methods have poor quality, more internal and surface defects, are prone to form carrier recombination centers, affect the transmission of electrons, lead to low electron transmission efficiency, and thus reduce the luminous performance of devices.

### Technical Solution

In view of this, the present disclosure provides a composite material, a preparation method thereof and a photoelectric device.

The present disclosure provides a composite material. The composite material includes an inorganic particle and a perfluorosulfonamide; wherein the inorganic particle is connected to the perfluorosulfonamide by a chemical bond, and a chemical formula of the perfluorosulfonamide is R—SO₂—NH₂, wherein R is selected from one or more of a perfluoroalkyl group, a perfluorocycloalkyl group, a perfluoroheterocycloalkyl group, and a perfluoroamine group.

Alternatively, a mole percentage of the perfluorosulfonamide in the composite material, is 15-25%.

The inorganic particle comprises one or more of oxygen and fluorine.

A material of the inorganic particle includes doped metal compound particle or non-doped metal compound particle.

Alternatively, the perfluoroalkyl group is a linear or branched perfluoroalkyl group with 1 to 10 carbon atoms in the main chain, a linear or branched perfluoroalkenyl group with 1 to 10 carbon atoms in the main chain, a linear or branched perfluoroalkynyl group with 1 to 10 carbon atoms in the main chain, or a combination of these groups.

The perfluorocycloalkyl group has 1 to 10 ring atoms.

The perfluoroheterocycloalkyl group has 1 to 10 ring atoms.

The perfluoroamine group has 1 to 10 carbon atoms in the main chain.

Alternatively, the perfluoroalkyl group is a linear or branched perfluoroalkyl group with 1 to 5 carbon atoms in the main chain, a linear or branched perfluoroalkenyl group with 1 to 5 carbon atoms in the main chain, a linear or branched perfluoroalkynyl group with 1 to 5 carbon atoms in the main chain, or a combination of these groups.

The perfluorocycloalkyl group has 1 to 5 ring atoms.

The perfluoroheterocycloalkyl group has 1 to 5 ring atoms.

The perfluoroamine group has 1 to 5 carbon atoms in the main chain.

Alternatively, the perfluorosulfonamide is selected from one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide.

A material of the non-doped metal compound particle is selected from one or more of ZnO, TiO₂, SnO, Ta₂O₃, CsF, LiF, CsCO₃, SnO₂, and ZrO₂.

A material of a metal compound particle in the doped metal compound particle is selected from one or more of ZnO, TiO₂, SnO, Ta₂O₃, CsF, LiF, CsCO₃, SnO₂, and ZrO₂, and a doping element in the doped metal compound particle is selected from one or more of aluminum, magnesium, lithium, manganese, yttrium, lanthanum, copper, nickel, zirconium, and cerium.

An average particle size of the inorganic particle is 5-10 nm.

The present disclosure also provides a preparation method of a composite material which includes following steps.

A metal hydroxide solution and a perfluorosulfonamide solution are provided, mixed, and reacted to obtain a perfluorosulfonamide metal salt.

An organic base and a polar solvent are provided, and the organic base, the polar solvent, and the perfluorosulfonamide metal salt are mixed to obtain the composite material, which includes an inorganic particle and a perfluorosulfonamide.

Alternatively, the metal hydroxide solution includes a metal hydroxide and a first solvent.

The perfluorosulfonamide solution includes perfluorosulfonamide and a fourth solvent.

Alternatively, the metal hydroxide is selected from one or more of zinc hydroxide, titanium hydroxide, tin hydroxide, tantalum hydroxide, aluminum hydroxide, cesium hydroxide, and lithium hydroxide.

The first solvent is selected from one or more of ethanol, ethylene glycol, butanol, glycerol, trimethoxybutanol, and methanol.

The perfluorosulfonamide is selected from one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide.

The fourth solvent is selected from one or more of ethanol, ethylene glycol, butanol, glycerol, trimethoxybutanol, and methanol.

The organic strong base is selected from one or more of a tetraalkyl-substituted quaternary ammonium base, a quaternary phosphonium base, and a guanidine compound.

The polar solvent is selected from tetrahydrofuran, ethanol, methanol, and isopropanol.

Alternatively, a concentration of the metal hydroxide in the metal hydroxide solution is 0.3-0.8 mmol/mL.

A concentration of the perfluorosulfonamide in the perfluorosulfonamide solution is 0.5-0.8 mmol/mL.

A molar ratio of the metal hydroxide in the metal hydroxide solution to the perfluorosulfonamide is (1-1.5):1.

A molar ratio of the perfluorosulfonamide metal salt to the organic base is (0.5-1):1.

Alternatively, a temperature for the reacting of the metal hydroxide solution and the perfluorosulfonamide solution is 40-60° C.

A time for the reacting of the metal hydroxide solution and the perfluorosulfonamide solution is 5-8 h.

A temperature for the mixing the organic base, the polar solvent and the perfluorosulfonamide metal salt is 30-50° C.

A time for the mixing the organic base, the polar solvent and the perfluorosulfonamide metal salt is 10-14 h.

Alternatively, a method for preparing the metal hydroxide solution including: providing an alkali solution and a metal salt solution, mixing and reacting to obtain the metal hydroxide solution.

Alternatively, the alkali solution includes an alkali and a second solvent.

The metal salt solution includes a metal salt and a third solvent.

Alternatively, the alkali is selected from one or more of sodium hydroxide, lithium hydroxide, potassium hydroxide.

The metal salt is selected from one or more of zinc salt, titanium salt, tin salt, tantalum salt, aluminum salt, cesium salt, lithium salt.

The second solvent is selected from ethanol, ethylene glycol, butanol, glycerol, trimethoxybutanol, and methanol.

The third solvent is selected from one or more of N,N-dimethylformamide, dimethyl sulfoxide, ethylene glycol monomethyl ether, ethylene glycol monoethyl ether, and ethylene glycol monobutyl ether.

Alternatively, a mixed time for the alkali solution and the metal salt solution is 2-8 h.

A molar ratio of the alkali to the metal salt is (1.1-1.3):1.

The present disclosure also provides a photoelectric device. The photoelectric device includes an anode, an emission layer, an electron functional layer and a cathode which are sequentially stacked, wherein a material of the electron functional layer includes the composite material, and the composite material includes an inorganic particle and a perfluorosulfonamide; wherein the inorganic particle is connected to the perfluorosulfonamide by a chemical bond, and a chemical formula of the perfluorosulfonamide is R—SO₂—NH₂, wherein R is selected from one or more of a perfluoroalkyl group, a perfluorocycloalkyl group, a perfluoroheterocycloalkyl group, and a perfluoroamine group.

Alternatively, a mole percentage of the perfluorosulfonamide in the composite material, is 15-25%.

The inorganic particle includes one or more of oxygen and fluorine.

A material of the inorganic particle includes doped metal compound particle or non-doped metal compound particle.

Alternatively, the perfluoroalkyl group is a linear or branched perfluoroalkyl group with 1 to 10 carbon atoms in the main chain, a linear or branched perfluoroalkenyl group with 1 to 10 carbon atoms in the main chain, a linear or branched perfluoroalkynyl group with 1 to 10 carbon atoms in the main chain, or a combination of these groups.

The perfluorocycloalkyl group has 1 to 10 ring atoms.

The perfluoroheterocycloalkyl group has 1 to 10 ring atoms.

The perfluoroamine group has 1 to 10 carbon atoms in the main chain.

Alternatively, the perfluorosulfonamide is selected from one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide.

A material of the non-doped metal compound particle is selected from one or more of ZnO, TiO₂, SnO, Ta₂O₃, CsF, LiF, CsCO₃, SnO₂, and ZrO₂.

A material of a metal compound particle in the doped metal compound particle is selected from one or more of ZnO, TiO₂, SnO, Ta₂O₃, CsF, LiF, CsCO₃, SnO₂, and ZrO₂, and a doping element in the doped metal compound particle is selected from one or more of aluminum, magnesium, lithium, manganese, yttrium, lanthanum, copper, nickel, zirconium, and cerium.

An average particle size of the inorganic particle is 5-10 nm.

A thickness of the electron functional layer is 15-30 nm.

Alternatively, the photoelectric device further includes a hole functional layer, located between the anode and the emission layer, and the hole functional layer includes one or more of a hole transport layer and a hole injection layer.

Alternatively, a material of the anode and the cathode is independently selected from one or more of indium doped tin oxide, fluorine doped tin oxide, antimony doped tin oxide, aluminum doped zinc oxide, gallium doped zinc oxide, indium doped zinc oxide, magnesium doped zinc oxide, aluminum doped magnesium oxide, AZO/Ag/AZO, AZO/AI/AZO, ITO/Ag/ITO, ITO/AI/ITO, ZnO/Ag/ZnO, ZnO/Al/ZnO, TiO₂/Ag/TiO₂, TiO₂/Al/TiO₂, ZnS/Ag/ZnS, ZnS/Al/ZnS, Ag, Al, Cu, Mo, Au, Pt, Si, Ca, Mg, and Ba.

A material of the emission layer includes one or more of 4,4′-Bis(N-carbazole)-1,1′-biphenyl: tris[2-(p-tolyl)pyridine iridium (III)], 4,4′,4″-tris(carbazole-9-yl)triphenylamine: tris[2-(p-tolyl)pyridine iridium], diaryl anthracene derivatives, stilbene aromatic derivatives, pyrene derivatives, fluorene derivatives, TBPe fluorescent materials, TTPX fluorescent materials, TBRb fluorescent materials, DBP fluorescent materials, delayed fluorescent materials, TTA materials, TADF materials, B—N covalent bond containing polymers, HLCT materials, Exciplex light-emitting materials, CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdZnSTe, CdHgSeS, CdHgSeTe, CdHgSTe, HgZnSeS, HgZnSeTe, HgZnSTe, SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, SnPbSTe, GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, CuInS₂, CuInSe₂, AgInS₂, CdSe/CdSeS/CdS, InP/ZnSeS/ZnS, CdZnSe/ZnSe/ZnS, CdSeS/ZnSeS/ZnS, CdSe/ZnS, CdSe/ZnSe/ZnS, ZnSe/ZnS, ZnSeTe/ZnS, CdSe/CdZnSeS/ZnS, InP/ZnSe/ZnS, AMX₃, and BMX₃; wherein A is a Cs⁺ ion, M is a divalent metal cation selected from one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, Eu²⁺, X is a halide anion selected from one or more of Cl⁻, Br⁻, I⁻; B is an organic amine cation selected from CH₃(CH₂)ₙ₋₂NH³⁺ or [NH₃(CH₂)ₙNH₃]²⁺ with n>2.

A material of the hole injection layer and the hole transport layer is each independently selected from one or more of 4,4′-N,N′-dicarbazolyl-biphenyl, N,N′-diphenyl-N,N′-bis(1-naphthyl)-1,1′-biphenyl-4,4″-diamine, N,N′-diphenyl-N,N′-bis(3-methylphenyl)-(1,1′-biphenyl)-4,4′-diamine, N,N′-bis(3-methylphenyl)-N,N′-bis(phenyl)-spiro, N,N′-di(4-(N,N′-diphenyl-amino)phenyl)-N,N′-diphenylbenzidine, 4,4′,4′-tris(N-carbazolyl)-triphenylamine, 4,4′,4′-tris(9H-carbazol-9-yl)triphenylamine, trichloroisocyanuric acid, terbium-doped phosphate-based green light emitting material, 2,3,6,7,10,11-hexacyano-1,4,5,8,9,12-hexaazatriphenylene, 4,4′,4′-tris(N-3-methylphenyl-N-phenylamino)triphenylamine, poly[(9,9′-dioctylfluorene-2,7-diyl)-co-(4,4′-(N-(4-sec-butyphenyl)diphenylamine))], poly(4-butylphenyl-diphenylamine), poly[bis(4-phenyl) (4-butylphenyl)amine], polyaniline, polypyrrole, poly(p-phenylenevinylene), polyphenylenevinylene, poly[2-methoxy-5-(2-ethylhexyloxy)-1,4-phenylenevinylene], poly[2-methoxy-5-(3′,7′-dimethyloctyloxy)-1,4-phenylenevinylene], copper phthalocyanine, aromatic tertiary amines, polynuclear aromatic tertiary amines, 4,4′-bis(carbazol-9-yl)-1,1′-biphenyl compounds, N,N,N′,N′-tetraarylbenzidines, PEDOT, PEDOT:PSS and derivatives thereof, PEDOT:PSS derivatives doped with s-MoO₃, poly(N-vinylcarbazole) and derivatives thereof, polymethacrylates and derivatives thereof, poly(9,9-octylfluorene) and derivatives thereof, poly(spirofluorene) and derivatives thereof, N,N′-di(naphthalen-1-yl)-N,N′-diphenylbenzidine, spiro-NPB, nanopolycrystalline diamond, microcrystalline cellulose, and tetracyanoquinodimethane, doped graphene, undoped graphene, transition metal oxides, transition metal sulfides, transition metal stannides.

The composite material includes an inorganic particle and a perfluorosulfonamide. Fluoride ions in the perfluorosulfonamide could be embedded into the surface of the inorganic particles. Because the fluoride ions in the perfluorosulfonamide have the same or similar ionic radius as fluoride ions and/or oxygen ions in the inorganic particles, lattice distortion of the inorganic particles caused by embedding the fluoride ions in the perfluorosulfonamide into the surface of the inorganic particles is small, thereby reducing scattering of free carriers, and further improving electron transport efficiency of the inorganic particles and light emitting performance of a device. In addition, the sulfonamide group in the perfluorosulfonamide has strong electronegativity, is easy to form a strong dipole effect with the inorganic particles, and is firmly combined on oxygen vacancies on the surface of the inorganic semiconductor nanoparticles, thereby reducing surface defects of the inorganic particles, inhibiting generation of non-radiative transitions, and improving stability of the inorganic particles. Furthermore, after the perfluorosulfonamide is combined with the inorganic particles, the inorganic particles could be inhibited from continuing to grow and agglomerate, thereby improving a storage time of the inorganic particles.

### BRIEF DESCRIPTION OF DRAWINGS

In order to more clearly explain the technical solutions in the embodiments of the present disclosure, the following will briefly introduce the drawings required in the description of the embodiments. Obviously, the drawings in the following description are only some embodiments of the present disclosure. For those skilled in the art, without paying any creative work, other drawings can be obtained based on these drawings.

FIG. 1 is a flowchart of a method for preparing a composite material according to an embodiment of the present disclosure.

FIG. 2 is a schematic diagram of the structure of a photoelectric device according to an embodiment of the present disclosure.

FIG. 3 is a schematic diagram of the structure of a photoelectric device according to another embodiment of the present disclosure.

FIG. 4 is a diagram showing the state of a composite material solution before being placed for seven days according to Example 1 and Comparative Example.

FIG. 5 is a diagram showing the state of a composite material solution after being placed for seven days according to Example 1 and Comparative Example.

In which, the reference numeral indicates:
photoelectric device 100; anode 10; emission layer 20; electron functional layer 30; cathode 40; hole functional layer 50.

### DETAILED DESCRIPTION

The technical solutions in the embodiments of the present disclosure will be clearly and completely described below with reference to the drawings in the embodiments of the present disclosure. Obviously, the described embodiments are only some of the embodiments of the present disclosure, rather than all of the embodiments. Based on the embodiments of the present disclosure, all other embodiments obtained by those skilled in the art without creative efforts are within the protection scope of the present disclosure.

The embodiments of the present disclosure provide a film and a preparation method thereof, and an optoelectronic device. The following respectively describes the details. It should be noted that the description order of the following embodiments is not used as a limitation on the preferred order of the embodiments. In addition, in the description of the present disclosure, the term “including” means “including but not limited to”. The terms first, second, third, etc. are only used as labels and do not impose numerical requirements or establish an order.

In this present disclosure, the “and/or” describes the association relationship of the associated objects, which means that there could be three kinds of relationships, for example, A and/or B could mean that A exists alone, A and B exist simultaneously, and B exists alone. Wherein A and B could be singular or plural.

In this present disclosure, the expressions such as “one or more” refer to one or more of the listed items, and “multiple” refers to any combination of two or more of the items, including any combination of a single item or multiple items. For example, “at least one of a, b or c” or “at least one of a, b, and c” could mean a, b, c, a-b (i.e., a and b), a-c, b-c, or a-b-c, where a, b, and c could be a single item or multiple items.

Various embodiments of the present disclosure could be presented in a range format. It should be understood that a range format is used for convenience and brevity, and should be understood flexibly to include numerical values explicitly specified as endpoints of the range. For instance, a range of “from 0.04 to 0.1” should be read to include the explicitly recited endpoints, 0.04 and 0.1, and all the individual intervening numbers between 0.04 and 0.1, such as 0.04, 0.05, 0.06, 0.07, 0.08, 0.09, and so forth. The same principles apply to ranges reciting one numerical value, a partial range, or several numerical values. Also, every numerical range given throughout this specification will include all the possible sub-ranges of the numerator values. For instance, every numerical range expressed in the form of “from X to Y” should be understood to include sub-ranges of the form “from X to Y-Z”, “from X-Y to Y”, and “from X-Y to Z”, where X<Y<Z. Also, every numerical range given throughout this specification will include any and all the fractional and integer values subsumed therein. For instance, every numerical range expressed in the form “from X to Y” should be understood to include any and all fractional and integer values subsumed therein, such as X, X 0.5, X 1, X 2, X 3, X 4, X 5, Y 1, Y 2, Y 3, Y 4, Y 5, and so forth.

In the present disclosure, perfluoro means that all the hydrogen atoms on the alkane, arene are replaced by halogen atoms. “Aryl or aromatic group” means that the aromatic hydrocarbon group derived from removing one hydrogen atom from the aromatic ring compound, could be monocyclic aryl, or fused ring aryl, or polycyclic aryl, and at least one of the polycyclic rings is an aromatic ring system. For example, “substituted or unsubstituted aryl having 6 to 40 ring atoms” means aryl containing 6 to 40 ring atoms, preferably substituted or unsubstituted aryl having 6 to 30 ring atoms, more preferably substituted or unsubstituted aryl having 6 to 18 ring atoms, particularly preferably substituted or unsubstituted aryl having 6 to 14 ring atoms, and the aryl is optionally further substituted; suitable examples include, but are not limited to, phenyl, biphenyl, terphenyl, naphthyl, anthryl, phenanthryl, fluoranthenyl, triphenylenyl, pyrenyl, perylenyl, tetracenyl, fluorenyl, perylenyl, acenaphthyl, and derivatives thereof. Understandably, multiple aryl groups could also be interrupted by short non-aromatic units (e.g. <10% non-H atoms, such as C, N or O atoms), specifically acenaphthene, fluorene, or 9,9-diarylfluorene, triarylamine, diaryl ether systems should also be included in the definition of aryl. The heteroatoms in the heterocycle are one or more of oxygen atoms, sulfur atoms, nitrogen atoms, phosphorus atoms.

In a first aspect, the present disclosure provides a composite material. The composite material includes an inorganic particle and a perfluorosulfonamide, wherein the inorganic particle is connected to the perfluorosulfonamide by a chemical bond, and a chemical formula of the perfluorosulfonamide is R—SO₂—NH₂, wherein R is selected from one or more of a perfluoroalkyl group, a perfluorocycloalkyl group, a perfluoroheterocycloalkyl group, and a perfluoroamine group.

The composite material includes the inorganic particle and the perfluorosulfonamide. The sulfonamide group in the perfluorosulfonamide has strong electronegativity, is more likely to form a strong dipole effect with the inorganic particle, and is firmly combined on the oxygen vacancies on the surface of the inorganic particle, so that the surface defects of the inorganic particle are reduced, the generation of non-radiative transition is inhibited, and the stability of the inorganic particle is improved. Furthermore, after the perfluorosulfonamide is combined with the inorganic particle, the inorganic particle is inhibited from continuing to grow and agglomerate, so that the storage time of the inorganic particle is prolonged.

In some embodiments, the perfluorosulfonamide has a structural formula as shown in formula (I).

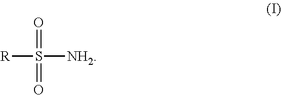

In some embodiments, the inorganic particle includes one or more of oxygen and fluorine. The fluorine ions in the perfluorosulfonamide could be embedded into the surface of the inorganic particle. Because the ionic radii of the fluorine ions in the perfluorosulfonamide are the same as or similar to the ionic radii of the fluorine ions and/or oxygen ions in the inorganic particle, the lattice distortion of the inorganic particle caused by the embedding of the fluorine ions in the perfluorosulfonamide into the surface of the inorganic particle is small, so that the scattering of electrons could be reduced, and the electron transport efficiency of the inorganic particle is improved.

In some embodiments, the perfluoroalkyl group could be a linear or branched perfluoroalkyl group with 1 to 10 carbon atoms in the main chain, a linear or branched perfluoroalkenyl group with 1 to 10 carbon atoms in the main chain, a linear or branched perfluoroalkynyl group with 1 to 10 carbon atoms in the main chain, or a combination of these groups.

In other embodiments, the perfluoroalkyl group could be a linear or branched perfluoroalkyl group with 1 to 5 carbon atoms in the main chain, a linear or branched perfluoroalkenyl group with 1 to 5 carbon atoms, a linear or branched perfluoroalkynyl group with 1 to 5 carbon atoms, or a combination of these groups.

In some embodiments, the perfluorocycloalkyl group could have 1 to 10 ring atoms.

In other embodiments, the perfluorocycloalkyl group could have 1 to 5 ring atoms.

In some embodiments, the perfluoroheterocycloalkyl group could have 1 to 10 ring atoms.

A heteroatom in the perfluoroheterocycloalkyl group could be one or more of an oxygen atom, a sulfur atom, a nitrogen atom, and a phosphorus atom.

In other embodiments, the perfluoroheterocycloalkyl group could have 1 to 5 ring atoms.

In some embodiments, the perfluoroamine group could have 1 to 10 carbon atoms in the main chain.

In other embodiments, the perfluoroamine group could have 1 to 5 carbon atoms in the main chain.

In the range of the number of carbon atoms in the main chain or the number of ring atoms, the R group has good stability and is not easy to break.

In some embodiments, a mole percentage of the perfluorosulfonamide in the composite material, is 15-25%, for example, 16-24%, 17-23%, 18-22%, 19-21%, etc. Within the mole range, the composite material has higher stability and lower resistance.

In some embodiments, the perfluorosulfonamide could be selected from, but not limited to, one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide.

In some embodiments, a material of the inorganic particle includes doped metal compound particle or non-doped metal compound particle.

A material of the non-doped metal compound particle is selected from one or more of ZnO, TiO₂, SnO, Ta₂O₃, CsF, LiF, CsCO₃, SnO₂, and ZrO₂.

A material of a metal compound particle in the doped metal compound particle is selected from one or more of ZnO, TiO₂, SnO, Ta₂O₃, CsF, LiF, CsCO₃, SnO₂, and ZrO₂, and a doping element in the doped metal compound particle is selected from one or more of aluminum, magnesium, lithium, manganese, yttrium, lanthanum, copper, nickel, zirconium, and cerium.

An average particle size of the inorganic particle is 5-10 nm, for example, 6-9 nm, 7-8 nm, etc.

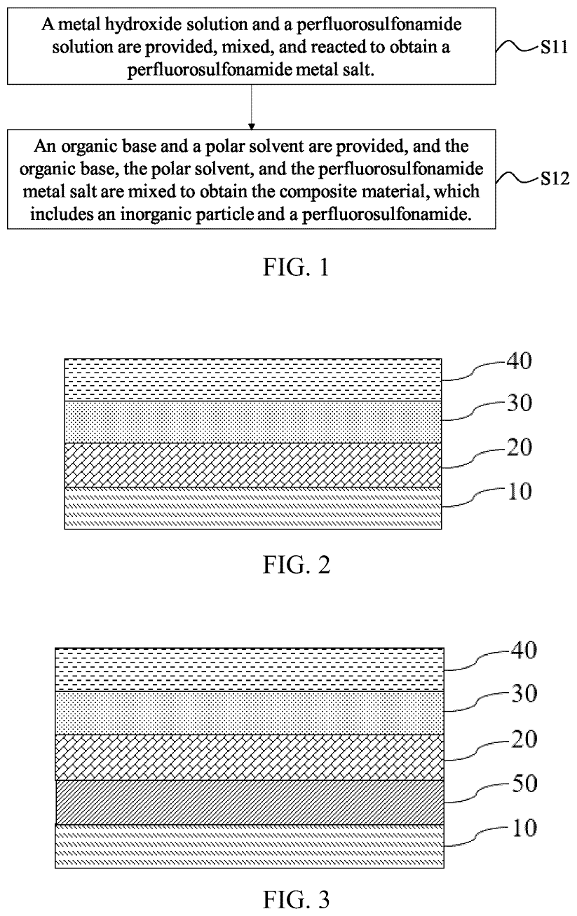

Referring to FIG. 1, the present disclosure also provides a preparation method of the composite material which includes steps S11-S12.

In step S11, a metal hydroxide solution and a perfluorosulfonamide solution are provided, mixed, and reacted to obtain a perfluorosulfonamide metal salt.

In step S12, an organic base and a polar solvent are provided, and the organic base, the polar solvent, and the perfluorosulfonamide metal salt are mixed to obtain the composite material, which includes an inorganic particle and a perfluorosulfonamide.

In some embodiments, the metal hydroxide solution includes a metal hydroxide and a first solvent.

In some embodiments, a concentration of the metal hydroxide in the metal hydroxide solution is 0.3-0.8 mmol/mL, for example, 0.4-0.6 mmol/mL, 0.5-0.7 mmol/mL, etc. Within the concentration range, the reaction rate could be promoted, and the metal hydroxide solution is prevented from hydrolyzing to generate a precipitate.

In some embodiments, the metal hydroxide is selected from one or more of zinc hydroxide, titanium hydroxide, tin hydroxide, tantalum hydroxide, aluminum hydroxide, cesium hydroxide, and lithium hydroxide.

In some embodiments, the first solvent is selected from one or more of ethanol, ethylene glycol, butanol, glycerol, trimethoxybutanol, and methanol.

In some embodiments, a method for preparing the metal hydroxide solution includes: an alkali solution and a metal salt solution are provided, mixed and reacted to obtain the metal hydroxide solution.

In some embodiments, the alkali solution includes an alkali and a second solvent.

In some embodiments, the metal salt solution includes a metal salt and a third solvent.

In some embodiments, a molar ratio of the alkali to the metal salt is (1.1-1.3):1, for example, could be (1.1-1.15):1, (1.15-1.2):1, (1.2-1.25):1, (1.25-1.3):1, etc. In the molar ratio range, the composite material has good dispersibility and high yield.

In some embodiments, the alkali could be selected from, but not limited to, one or more of sodium hydroxide, lithium hydroxide, potassium hydroxide.

In some embodiments, the metal salt is selected from one or more of zinc salt, titanium salt, tin salt, tantalum salt, aluminum salt, cesium salt, lithium salt. Further, the zinc salt could be selected from, but not limited to, one or more of zinc acetate, zinc citrate, zinc lactate.

In some embodiments, a mixed time for the alkali solution and the metal salt solution is 2-8 h, for example, could be 3-6 h, 4-7 h, 5-8 h, etc. In the mixed time range, the reaction could be sufficient, and the metal hydroxide solution is ensured to be not turbid.

In some embodiments, the second solvent could be selected from, but not limited to, one or more of ethanol, ethylene glycol, butanol, glycerol, trimethoxybutanol, and methanol.

In some embodiments, the third solvent could be selected from, but not limited to, one or more of N,N-dimethylformamide (DMF), dimethyl sulfoxide (DMSO), ethylene glycol monomethyl ether, ethylene glycol monoethyl ether, and ethylene glycol monobutyl ether.

In some embodiments, the perfluorosulfonamide solution includes perfluorosulfonamide and a fourth solvent.

The perfluorosulfonamide is as described above. The fourth solvent could be selected from, but not limited to, one or more of ethanol, ethylene glycol, butanol, glycerol, trimethoxybutanol, and methanol.

In some embodiments, a molar ratio of the metal hydroxide in the metal hydroxide solution to the perfluorosulfonamide is (1-1.5):1, for example, could be (1-1.1):1, (1.1-1.2):1, (1.2-1.3):1, (1.3-1.4):1, (1.4-1.5):1, etc. In the molar ratio range, the prepared composite material has high stability and low resistance.

In some embodiments, a concentration of the perfluorosulfonamide in the perfluorosulfonamide solution is 0.5-0.8 mmol/mL, for example, could be 0.5-0.55 mmol/mL, 0.55-0.6 mmol/mL, 0.6-0.65 mmol/mL, 0.65-0.7 mmol/mL, 0.7-0.75 mmol/mL, 0.75-0.8 mmol/mL, etc. The concentration range is matched with the concentration of the metal hydroxide solution, and could improve the performance of the prepared composite material.

In some embodiments, the process of the reaction further includes heating, and a temperature for the heating is 40-60° C., for example, could be 41-59° C., 42-58° C., 43-57° C., 44-56° C., 45-55° C., 46-54° C., 47-53° C., 48-52° C., 49-51° C., etc. In the range of the temperature of the heating, the reaction rate is stable, and the preparation of the composite material could be promoted.

In some embodiments, a time for the heating is 5-8 h, for example, could be 5-6 h, 6-7 h, 7-8 h, etc. In the range of the time of the heating, the prepared composite material has good dispersibility, is not easy to agglomerate, and has a suitable yield.

In some embodiments, a molar ratio of the perfluorosulfonamide metal salt to the organic base is (0.5-1):1, for example, could be (0.5-0.6):1, (0.6-0.7):1, (0.7-0.8):1, (0.8-0.9):1, (0.9-1):1, etc. In the molar ratio range, the reaction could be promoted sufficiently, and the prepared composite material has good dispersibility.

In some embodiments, a concentration of the organic base in the polar solvent is 0.5-0.9 mmol/mL, for example, could be 0.5-0.6 mmol/mL, 0.6-0.7 mmol/mL, 0.7-0.8 mmol/mL, 0.8-0.9 mmol/mL, etc. The concentration range is matched with the concentration of the perfluorosulfonamide solution, and could improve the performance of the prepared composite material.

In some embodiments, a pH of the organic base is 12-14, in other words, the organic base is an organic strong base.

In some embodiments, the organic strong base could be selected from, but is not limited to, one or more of a tetraalkyl-substituted quaternary ammonium base, a quaternary phosphonium base, and a guanidine compound.

In some embodiments, a hydrocarbon group in the tetraalkyl-substituted quaternary ammonium base is a linear aliphatic hydrocarbon with 1-4 carbon atoms. Exemplarily, the tetraalkyl-substituted quaternary ammonium base could be one or more of tetramethylammonium hydroxide, tetraethylammonium hydroxide, tetrapropylammonium hydroxide, and tetrabutylammonium hydroxide.

In some embodiments, the polar solvent could be selected from, but is not limited to, one or more of tetrahydrofuran, ethanol, methanol, and isopropanol.

In some embodiments, a temperature for the mixing the organic base, the polar solvent and the perfluorosulfonamide metal salt is 30-50° C., for example, 31-49° C., 32-48° C., 33-47° C., 34-46° C., 35-45° C., 36-44° C., 37-43° C., 38-42° C., 39-41° C., etc. The rate is stable in the mixing temperature range, which could promote the preparation of the composite material.

In some embodiments, a time for the mixing the organic base, the polar solvent and the perfluorosulfonamide metal salt is 10-14 h, for example, 10-11 h, 11-12 h, 12-13 h, 13-14 h, etc. The composite material prepared in the mixing time range has good dispersibility, is not easy to agglomerate and has a suitable yield.

In some embodiments, a molar percentage of the perfluorosulfonamide in the composite material is 15-25%, for example, 16-24%, 17-23%, 18-22%, 19-21%, etc. In the molar range, the perfluorosulfonamide could effectively improve the stability of the inorganic particle, inhibit the continuous growth and agglomeration of the inorganic particle and improve the storage time of the inorganic particle after being combined with the inorganic particle.

The method for preparing the composite material introduces the perfluorosulfonamide into the inorganic particle. The fluorine ions in the perfluorosulfonamide could be embedded into the surface of the inorganic particle. Because the ion radius of the fluorine ions in the perfluorosulfonamide is the same as or similar to that of the fluorine ions and/or oxygen ions in the inorganic particle, the lattice distortion of the inorganic particle caused by the embedding of the fluorine ions in the perfluorosulfonamide into the surface of the inorganic particle is small, thereby reducing the scattering of free carriers and improving the electron transport efficiency of the inorganic particle. In addition, the sulfonylamide group in the perfluorosulfonamide has strong electronegativity, is easy to form a strong dipole effect with the inorganic particle, and is firmly combined on the oxygen vacancies on the surface of the inorganic semiconductor nanoparticles, thereby reducing the surface defects of the inorganic particle, inhibiting the generation of non-radiative transitions, and improving the stability of the inorganic particle. Furthermore, the perfluorosulfonamide could inhibit the continuous growth and agglomeration of the inorganic particle after being combined with the inorganic particle, thereby improving the storage time of the inorganic particle. In summary, the method for preparing the composite material is simple in operation, low in cost, and capable of preparing the composite material with few surface defects, high conductivity and strong stability.

Referring to FIG. 2, the present disclosure further provides a photoelectric device 100. The photoelectric device 100 includes an anode 10, an emission layer 20, an electron functional layer 30 and a cathode 40 which are sequentially stacked, wherein a material of the electron functional layer 30 includes the composite material described above, and the electron functional layer 30 includes one or more of an electron injection layer and an electron transport layer.

In some embodiments, a thickness of the electron functional layer 30 is 15-30 nm, for example, could be 16-29 nm, 17-28 nm, 18-27 nm, 18-26 nm, 19-25 nm, 20-24 nm, 21-23 nm, 22-23 nm, etc. In this thickness range, the electron transport efficiency and hole transport efficiency in the photoelectric device 100 could be well matched, which could effectively promote the recombination of electrons and holes, thereby improving the luminous performance of the photoelectric device 100.

Referring further to FIG. 3, in some embodiments, the photoelectric device 100 further includes a hole functional layer 50, and the hole functional layer 50 includes one or more of a hole transport layer and a hole injection layer.

In some embodiments, a material of the anode 10 and the cathode 40 could be electrodes known in the art for photoelectric devices, for example, could be independently selected from, but are not limited to, one or more of a metal, a carbon material, and a metal oxide. The metal includes one or more of Al, Ag, Cu, Mo, Au, Ba, Ca, Yb, and Mg; the carbon material includes one or more of graphite, carbon nanotube, graphene, and carbon fiber; the metal oxide includes a doped or undoped metal oxide, including one or more of ITO, FTO, ATO, AZO, GZO, IZO, MZO, and AMO, or a composite electrode including a metal sandwiched between doped or undoped transparent metal oxides, including one or more of AZO/Ag/AZO, AZO/AI/AZO, ITO/Ag/ITO, ITO/AI/ITO, ZnO/Ag/ZnO, ZnO/Al/ZnO, ZnS/Ag/ZnS, ZnS/Al/ZnS, TiO₂/Ag/TiO₂, and TiO₂/Al/TiO₂. Herein, “/” represents a stacked structure, for example, AZO/Ag/AZO represents a composite electrode including an AZO layer, an Ag layer, and an AZO layer stacked in sequence.

In some embodiments, a material of the emission layer 20 could be selected from, but is not limited to, one or more of an organic luminous material and a quantum dot luminous material.

The organic luminous material could be selected from, but not limited to, one or more of CBP:Ir(mppy)₃ (4,4′-bis(N-carbazole)-1,1′-biphenyl: tris[2-(p-tolyl)pyridine iridium (III)]), TCTX:Ir(mmpy) (4,4′,4″-tris(carbazol-9-yl)triphenylamine: tris[2-(p-tolyl)pyridine iridium]), diaryl anthracene derivatives, stilbene aromatic derivatives, pyrene derivatives, fluorene derivatives, TBPe fluorescent materials, TTPX fluorescent materials, TBRb fluorescent materials, DBP fluorescent materials, delayed fluorescent materials, TTA materials, TADF (thermally activated delayed) materials, B—N covalently bonded containing polymers, HLCT (hybrid local charge transfer excited state) materials, Exciplex (exciplex) luminous materials.

The quantum dot luminous material could be selected from, but not limited to, one or more of a single-structure quantum dot, a core-shell structure quantum dot, and a perovskite type semiconductor material.

A material of the single-structure quantum dot, a core material of the core-shell structure quantum dot and a shell material of the core-shell structure quantum dot could be respectively selected from, but not limited to, one or more of the II-VI compound, the IV-VI compound, the III-V compound and the I-III-VI compound. The II-VI compound could be selected from, but not limited to, one or more of CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdZnSTe, CdHgSeS, CdHgSeTe, CdHgSTe, HgZnSeS, HgZnSeTe and HgZnSTe. The IV-VI compound could be selected from, but not limited to, one or more of SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, SnPbSTe. The III-V compound could be selected from, but not limited to, one or more of GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GalInPSb, InAlNP, InAlNAs, InAlNSb, InAlPAs and InAlPSb. The I-III-VI compound could be selected from, but not limited to, one or more of CuInS₂, CuInSe₂ and AgInS₂.

As an example, the core-shell structured quantum dot could be selected from, but not limited to, one or more of CdSe/CdSeS/CdS, InP/ZnSeS/ZnS, CdZnSe/ZnSe/ZnS, CdSeS/ZnSeS/ZnS, CdSe/ZnS, CdSe/ZnSe/ZnS, ZnSe/ZnS, ZnSeTe/ZnS, CdSe/CdZnSeS/ZnS, and InP/ZnSe/ZnS.

The perovskite semiconductor material could be selected from, but not limited to, a doped or non-doped inorganic perovskite semiconductor, or an organic-inorganic hybrid perovskite semiconductor. The inorganic perovskite semiconductor has a general structure of AMX₃, wherein A is a Cs⁺ ion, M is a divalent metal cation selected from one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, Eu²⁺, and X is a halide anion selected from one or more of Cl⁻, Br⁻, I⁻. The organic-inorganic hybrid perovskite semiconductor has a general structure of BMX₃, wherein B is an organic amine cation selected from CH₃(CH₂)ₙ₋₂NH³⁺ or [NH₃(CH₂)ₙNH₃]²⁺ with n>2, Mis a divalent metal cation selected from one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, Eu²⁺, and X is a halide anion selected from one or more of Cl⁻, Br⁻, I⁻.

In some embodiments, a material of the hole injection layer 60 and the hole transport layer 50 could be each independently selected from, but are not limited to, one or more of 4,4′-N,N′-dicarbazolyl-biphenyl (CBP), N,N′-diphenyl-N,N′-bis(1-naphthyl)-1,1′-biphenyl-4,4″-diamine (a-NPD), N,N′-diphenyl-N,N′-bis(3-methylphenyl)-(1,1′-biphenyl)-4,4′-diamine (TPD), N,N′-bis(3-methylphenyl)-N,N′-bis(phenyl)-spiro(spiro-TPD), N,N′-di(4-(N,N′-diphenyl-amino)phenyl)-N,N′-diphenylbenzidine (DNTPD), 4,4′,4′-tris(N-carbazolyl)-triphenylamine (TCTA), 4,4′,4′-tris(9H-carbazol-9-yl)triphenylamine (TCATA), trichloroisocyanuric acid (TCCA), terbium-doped phosphate-based green light emitting material (TAPC), 2,3,6,7,10,11-hexacyano-1,4,5,8,9,12-hexaazatriphenylene (HAT-CN), 4,4′,4′-tris(N-3-methylphenyl-N-phenylamino)triphenylamine (m-MTDATA), poly[(9,9′-dioctylfluorene-2,7-diyl)-co-(4,4′-(N-(4-sec-butyphenyl)diphenylamine))] (TFB), poly(4-butylphenyl-diphenylamine) (poly-TPD), poly[bis(4-phenyl) (4-butylphenyl)amine] (Poly-TPD), polyaniline, polypyrrole, poly(p-phenylenevinylene), polyphenylenevinylene (PPV), poly[2-methoxy-5-(2-ethylhexyloxy)-1,4-phenylenevinylene] (MEH-PPV), poly[2-methoxy-5-(3′,7′-dimethyloctyloxy)-1,4-phenylenevinylene] (MOMO-PPV), copper phthalocyanine, aromatic tertiary amines, polynuclear aromatic tertiary amines, 4,4′-bis(carbazol-9-yl)-1,1′-biphenyl compounds, N,N,N′,N′-tetraarylbenzidines, PEDOT, PEDOT:PSS and derivatives thereof, PEDOT:PSS derivatives doped with s-MoO₃ (PEDOT:PSS:s-MoO₃), poly(N-vinylcarbazole) (PVK) and derivatives thereof, polymethacrylates and derivatives thereof, poly(9,9-octylfluorene) and derivatives thereof, poly(spirofluorene) and derivatives thereof, N,N′-di(naphthalen-1-yl)-N,N′-diphenylbenzidine (NPB), spiro-NPB, nanopolycrystalline diamond, microcrystalline cellulose (MCC), and tetracyanoquinodimethane (F4-TCQN), doped graphene, undoped graphene, transition metal oxides, transition metal sulfides, transition metal stannides.

The photoelectric device uses a composite material of inorganic particle and perfluorosulfonamide as the material of the electron functional layer, the composite material has few surface defects, high conductivity and strong stability, could promote the transmission of electrons, and thus improves the luminous efficiency and stability of the photoelectric device.

The present disclosure also provides a display device, which includes the photoelectric device.

The display device could be any electronic product with a display function, and the electronic product includes, but is not limited to, a smartphone, a tablet computer, a notebook computer, a digital camera, a digital video camera, a smart wearable device, a smart weighing electronic scale, a vehicle-mounted display, a television, or an electronic book reader, wherein the smart wearable device could be, for example, a smart bracelet, a smart watch, a virtual reality (VR) helmet, or the like.

The present disclosure is described in detail below with reference to specific examples. The following examples are only part of the present disclosure and are not intended to limit the present disclosure.

### Example 1

This example provides a composite material and a preparation method thereof. The composite material includes zinc oxide nanoparticles and perfluorooctyl sulfonamide connected to the zinc oxide nanoparticles, wherein a molar ratio of the perfluorooctyl sulfonamide is 25%, and the preparation method is as follows.

5000 mg of zinc acetate was dissolved in 90 mL of DMSO solution, 710 mg of lithium hydroxide was dissolved in 90 mL of ethanol solution, the two solutions were mixed, stirred for 60 min, then transferred to a 250 mL flask, reacted at room temperature for 3 h, naturally cooled, washed with ethyl acetate and n-heptane three times respectively, and dissolved in butanol solvent to prepare a 0.5 mmol/mL zinc hydroxide hydrate solution.

12.47 g of perfluorooctyl sulfonamide was dissolved in 50 mL of ethanol, mixed with 50 mL of the 0.5 mmol/mL zinc hydroxide hydrate, reacted at 40° C. for 5 h, the solvent was removed after the reaction, washed with ice water, filtered, and perfluorooctyl sulfonamide zinc was obtained.

The perfluorooctyl sulfonamide zinc and 25 mmol of tetramethylammonium hydroxide were dissolved in 50 mL of tetrahydrofuran solvent, reacted at 50° C. for 10 h, the solvent was removed after the reaction, washed, and the composite material was obtained.

### Example 2

The example is basically the same as Example 1, except that the perfluorooctyl sulfonamide is replaced with N-ethyl perfluoro-n-octanesulfonamide.

### Example 3

The example is basically the same as Example 1, except that the perfluorooctyl sulfonamide is replaced with perfluoroethanesulfonamide.

### Example 4

The example is basically the same as Example 1, except that the mass of the perfluorooctyl sulfonamide is 10.23 g, and the molar ratio of the perfluorooctyl sulfonamide in the obtained product is 20%.

### Example 5

This example is basically the same as Example 1, except that the mass of the perfluorooctanesulfonamide is 8.29 g and the mole ratio of the perfluorooctanesulfonamide in the product is 15%.

### Example 6

This example is basically the same as Example 1, except that the concentration of the hydrate of zinc hydroxide is 0.3 mmol/mL.

### Example 7

This example is basically the same as Example 1, except that the concentration of the hydrate of zinc hydroxide is 0.8 mmol/mL.

### Example 8

This example is basically the same as Example 1, except that the volume of the ethanol solvent for 12.47 g of the perfluorooctanesulfonamide is 38.46 mL.

### Example 9

This example is basically the same as Example 1, except that the volume of the ethanol solvent for 12.47 g of the perfluorooctanesulfonamide is 31.25 mL.

### Example 10

This example is basically the same as Example 1, except that the tetramethylammonium hydroxide is 33 mmoL.

### Example 11

This example is basically the same as Example 1, except that the tetramethylammonium hydroxide is 50 mmoL.

### Example 12

This example is basically the same as Example 1, except that the 25 mmol of the tetramethylammonium hydroxide is dissolved in 35.71 mL of the tetrahydrofuran solvent.

### Example 13

This example is basically the same as Example 1, except that the 25 mmol of the tetramethylammonium hydroxide is dissolved in 27.78 mL of the tetrahydrofuran solvent.

### Example 14

This example is basically the same as Example 1, except that the reaction temperature of the perfluorooctanesulfonamide solution and the hydrate solution of zinc hydroxide is 50° C.

### Example 15

This example is basically the same as Example 1, except that the reaction temperature of the perfluorooctanesulfonamide solution and the hydrate solution of zinc hydroxide is 60° C.

### Example 16

This example is basically the same as Example 1, except that the reaction temperature of the perfluorooctanesulfonamide zinc and the tetramethylammonium hydroxide is 30° C.

### Example 17

This example is basically the same as Example 1, except that the reaction temperature of the perfluorooctanesulfonamide zinc and the tetramethylammonium hydroxide is 40° C.

### Example 18

This example is basically the same as Example 1, except that the mass of the lithium hydroxide is 783 mg.

### Example 19

This example is basically the same as Example 1, except that the mass of the lithium hydroxide is 848 mg.

### Example 20

This example is basically the same as Example 1, except that the zinc oxide nanoparticles in this example are doped with magnesium.

### Example 21

This example is basically the same as Example 1, except that the zinc oxide nanoparticles in this example are replaced with TiO₂ nanoparticles.

### Comparative Example

This comparative example is basically the same as Example 1, except that the electron transport material in this comparative example does not contain perfluorooctyl sulfonamide. Composite material stability test

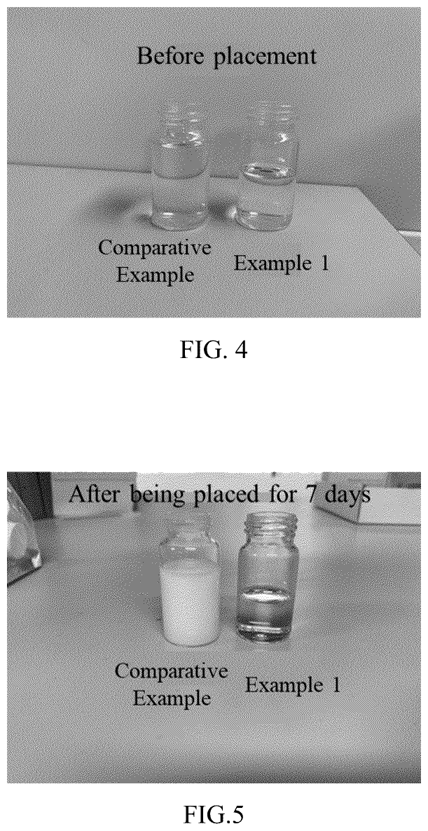

The composite materials prepared in Examples 1-21 and Comparative Example were tested for particle size before and after being placed for 7 days using a particle size tester, and the results are shown in Table 1. The solution states of the composite materials prepared in Example 1 and Comparative Example before and after being placed for 7 days were observed, and the comparison diagrams are shown in FIG. 4 and FIG. 5.

TABLE 1

| | Before placement | After placement |
|---|---|---|
| Composite material | (nm) | (nm) |
| Example 1 | 4.7 | 6.5 |
| Example 2 | 4.5 | 6.3 |
| Example 3 | 4.6 | 6.8 |
| Example 4 | 4.9 | 7.4 |
| Example 5 | 4.8 | 8.5 |
| Example 6 | 4.6 | 7.5 |
| Example 7 | 4.9 | 7.6 |
| Example 8 | 4.9 | 8.3 |
| Example 9 | 5.2 | 8.1 |
| Example 10 | 5.6 | 8.0 |
| Example 11 | 4.8 | 7.6 |
| Example 12 | 5.9 | 7.9 |
| Example 13 | 4.9 | 8.0 |
| Example 14 | 5.5 | 8.8 |
| Example 15 | 4.9 | 9.1 |
| Example 16 | 5.6 | 7.8 |
| Example 17 | 5.4 | 7.6 |
| Example 18 | 5.3 | 8.6 |
| Example 19 | 4.9 | 7.5 |
| Example 20 | 4.1 | 7.2 |
| Example 21 | 4.6 | 6.8 |
| Comparative Example | 5.7 | 1058 |

As shown in Table 1, after being placed for 7 days, the particle sizes of the composite materials in Examples 1-21 slightly increased, while the particle size of the composite material in Comparative Example significantly increased, indicating that the composite material in Comparative Example had serious agglomeration, while the composite material added with perfluorosulfonamide had good dispersibility, further indicating that the perfluorosulfonamide could reduce the continued growth and agglomeration of inorganic particle and improve the preservation time of the inorganic particle.

As shown in FIG. 4 and FIG. 5, the solutions of the composite materials of Example 1 and the Comparative Example are both clear and transparent before being placed, and there is no obvious difference. However, after being placed at room temperature for 7 days, the solution of the composite material of Example 1 has no obvious change, and the solution is clear and transparent, while the solution of the composite material of the Comparative Example is obviously turbid, the composite material is agglomerated, and the transparency is very low. It is shown that the composite material of Example 1 still maintains good dispersibility, and the addition of the perfluorosulfonamide effectively inhibits the agglomeration of the inorganic particle and improves the stability of the inorganic particle.

### Device Example 1

This example provides a quantum dot light emitting diode, and a preparation method thereof is as follows.

A patterned ITO substrate is sequentially ultrasonically cleaned with acetone, a detergent, deionized water and isopropyl alcohol, each ultrasonic cleaning lasting for 15 minutes, the cleaned ITO substrate is dried, the ITO substrate surface is treated with ultraviolet-ozone for 5 minutes, and the anode 10 with a thickness of 20 nm is formed.

PEDOT:PSS is deposited on the anode 10, heated at 150° C. for 30 minutes to remove moisture, and a hole injection layer 60 with a thickness of 30 nm is formed.

The ITO substrate coated with the hole injection layer 60 is placed in a nitrogen atmosphere, TFB is deposited, heated at 150° C. for 30 minutes to remove solvent, and a hole transport layer 50 with a thickness of 30 nm is formed.

CdSe/ZnS quantum dot material is deposited on the hole transport layer 50, heated at 100° C. for 10 minutes to remove solvent, and a quantum dot light emitting layer 20 with a thickness of 30 nm is formed.

The composite material of Example 1 is deposited on the quantum dot light emitting layer 20, annealed at 80° C. for 30 minutes, and an electron transport layer 30 with a thickness of 30 nm is formed.

Ag with a thickness of 80 nm is evaporated on the electron transport layer 30 to form a cathode 40.

The above basic device is packaged by using ultraviolet curing glue for 3 minutes to obtain a quantum dot light emitting diode.

### Device Examples 2-21

Device Examples 2-21 are basically the same as Device Example 1, except that the composite material of Example 1 is replaced by the composite material of Examples 2-21 respectively.

### Device Example 22

The device example is basically the same as Device Example 1, except that the thickness of the electron transport layer in the device example is 20 nm.

### Device Example 23

The device example is basically the same as Device Example 1, except that the thickness of the electron transport layer in the device example is 15 nm.

### Device Comparative Example

The device comparative example is basically the same as Device Example 1, except that the composite material of Example 1 is replaced by the material of the comparative example.

The quantum dot light emitting diodes of Device Examples 1-23 and the Comparative Example were subjected to external quantum efficiency (EQE) testing, wherein the testing method was as follows: the ratio of the number of electron-hole pairs injected into the quantum dots to the number of emitted photons, in percentage, was an important parameter for measuring the quality of the electroluminescent device, and could be obtained by using an EQE optical testing instrument. The specific calculation formula was as follows.

$$E Q E = η e η r χ \frac{{K}_{R}}{{K}_{R} + {K}_{NR}} .$$

wherein, ne is the light output coupling efficiency, nr is the ratio of the number of carriers recombined to the number of injected carriers, x is the ratio of the number of excitons generating photons to the total number of excitons, KR is the radiation process rate, and KNR is the non-radiation process rate.

Test conditions: performed at room temperature, air humidity was 30-60%.

A maximum brightness of the quantum dot light emitting diodes was tested by using a brightness meter PR650.

The test results of the external quantum efficiency (EQE) and the maximum brightness are shown in Table 2.

TABLE 2

| | External | |
|---|---|---|
| | quantum | Maximum |
| | efficiency | brightness |
| Composite material | (EQE) (%) | (nit) |
| Device Example 1 | 15.4 | 3250 |
| Device Example 2 | 15.3 | 3228 |
| Device Example 3 | 15.5 | 3271 |
| Device Example 4 | 15.6 | 3292 |
| Device Example 5 | 14.7 | 3102 |
| Device Example 6 | 14.2 | 2996 |
| Device Example 7 | 14.7 | 3102 |
| Device Example 8 | 15.3 | 3228 |
| Device Example 9 | 15.0 | 3165 |
| Device Example 10 | 15.5 | 3271 |
| Device Example 11 | 14.9 | 3144 |
| Device Example 12 | 14.8 | 3123 |
| Device Example 13 | 14.9 | 3144 |
| Device Example 14 | 15.4 | 3249 |
| Device Example 15 | 15.3 | 3228 |
| Device Example 16 | 15.3 | 3228 |
| Device Example 17 | 15.4 | 3249 |
| Device Example 18 | 15.0 | 3165 |
| Device Example 19 | 14.8 | 3123 |
| Device Example 20 | 15.5 | 3271 |
| Device Example 21 | 14.4 | 3038 |
| Device Comparative | 15.1 | 3186 |
| Example | | |

As shown in Table 2, compared with the photoelectric device of the Device Comparative Example, the photoelectric devices of the Device Examples 1-23 have higher external quantum efficiency and larger maximum brightness, which indicates that adding the perfluorosulfonamide as the electron transport layer material in the inorganic particle could effectively improve the luminous efficiency of the photoelectric device and improve the performance of the photoelectric device. This is because the F ion radius in the perfluorosulfonamide is close to the O ion radius, the fluorine ion replaces the oxygen ion in the zinc oxide, the lattice distortion generated in the zinc oxide crystal is small, the scattering of the free carriers is reduced, and the electron transport efficiency is improved. In addition, the perfluorosulfonamide has strong electronegativity, could more easily form a strong dipole effect with the metal ions on the surface of the inorganic particle, and could more easily combine the sulfonamide group on the oxygen vacancy, thereby reducing the surface defects of the inorganic particle, inhibiting the generation of non-radiative transition, maintaining the stability of the inorganic particle, and improving the performance of the photoelectric device.

Composite material, preparation method thereof and photoelectric device are described in detail above. The principles and embodiments of the present disclosure have been described with reference to specific embodiments, and the description of the above embodiments is merely intended to aid in the understanding of the method of the present disclosure and its core idea. At the same time, changes may be made by those skilled in the art to both the specific implementations and the scope of present disclosure in accordance with the teachings of the present disclosure. In view of the foregoing, the content of the present specification should not be construed as limiting the disclosure.

1. A composite material, comprising:
an inorganic particle; and
a perfluorosulfonamide; wherein the inorganic particle is connected to the perfluorosulfonamide by a chemical bond, and a chemical formula of the perfluorosulfonamide is R—SO₂—NH₂, wherein R is selected from one or more of a perfluoroalkyl group, a perfluorocycloalkyl group, a perfluoroheterocycloalkyl group, and a perfluoroamine group.

2. The composite material according to claim 1, wherein a mole percentage of the perfluorosulfonamide in the composite material, is 15-25%;
the inorganic particle comprises one or more of oxygen and fluorine; and
a material of the inorganic particle comprises doped metal compound particle or non-doped metal compound particle.

3. The composite material according to claim 1, wherein the perfluoroalkyl group is a linear or branched perfluoroalkyl group with 1 to 10 carbon atoms in the main chain, a linear or branched perfluoroalkenyl group with 1 to 10 carbon atoms in the main chain, a linear or branched perfluoroalkynyl group with 1 to 10 carbon atoms in the main chain, or a combination of these groups;
the perfluorocycloalkyl group has 1 to 10 ring atoms;
the perfluoroheterocycloalkyl group has 1 to 10 ring atoms; and
the perfluoroamine group has 1 to 10 carbon atoms in the main chain.

4. The composite material according to claim 3, wherein the perfluoroalkyl group is a linear or branched perfluoroalkyl group with 1 to 5 carbon atoms in the main chain, a linear or branched perfluoroalkenyl group with 1 to 5 carbon atoms in the main chain, a linear or branched perfluoroalkynyl group with 1 to 5 carbon atoms in the main chain, or a combination of these groups;
the perfluorocycloalkyl group has 1 to 5 ring atoms;
the perfluoroheterocycloalkyl group has 1 to 5 ring atoms; and
the perfluoroamine group has 1 to 5 carbon atoms in the main chain.

5. The composite material according to claim 2, wherein the perfluorosulfonamide is selected from one or more of perfluorooctylsulfonamide, N-ethyl perfluoro-n-octanesulfonamide, perfluoroethanesulfonamide, perfluorocyclopentanesulfonamide, and perfluorocyclohexanesulfonamide, perfluorobutanesulfonamide;
a material of the non-doped metal compound particle is selected from one or more of ZnO, TiO₂, SnO, Ta₂O₃, CsF, LiF, CsCO₃, SnO₂, and ZrO₂; a material of a metal compound particle in the doped metal compound particle is selected from one or more of ZnO, TiO₂, SnO, Ta₂O₃, CsF, LiF, CsCO₃, SnO₂, and ZrO₂, and a doping element in the doped metal compound particle is selected from one or more of aluminum, magnesium, lithium, manganese, yttrium, lanthanum, copper, nickel, zirconium, and cerium; and/or
an average particle size of the inorganic particle is 5-10 nm.

6. A preparation method of a composite material, comprising:
providing a metal hydroxide solution and a perfluorosulfonamide solution, mixing and reacting to obtain a perfluorosulfonamide metal salt; and
providing an organic base and a polar solvent, and mixing the organic base, the polar solvent and the perfluorosulfonamide metal salt to obtain the composite material, wherein the composite material comprises an inorganic particle and perfluorosulfonamide.

7. The preparation method according to claim 6, wherein the metal hydroxide solution comprises a metal hydroxide and a first solvent; and/or
the perfluorosulfonamide solution comprises perfluorosulfonamide and a fourth solvent.

8. The preparation method according to claim 7, wherein the metal hydroxide is selected from one or more of zinc hydroxide, titanium hydroxide, tin hydroxide, tantalum hydroxide, aluminum hydroxide, cesium hydroxide, and lithium hydroxide;
the first solvent is selected from one or more of ethanol, ethylene glycol, butanol, glycerol, trimethoxybutanol, and methanol;
the perfluorosulfonamide is selected from one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide;
the fourth solvent is selected from one or more of ethanol, ethylene glycol, butanol, glycerol, trimethoxybutanol, and methanol;
the organic strong base is selected from one or more of a tetraalkyl-substituted quaternary ammonium base, a quaternary phosphonium base, and a guanidine compound; and
the polar solvent is selected from tetrahydrofuran, ethanol, methanol, and isopropanol.

9. The preparation method according to claim 7, wherein a concentration of the metal hydroxide in the metal hydroxide solution is 0.3-0.8 mmol/mL;
a concentration of the perfluorosulfonamide in the perfluorosulfonamide solution is 0.5-0.8 mmol/mL;
a molar ratio of the metal hydroxide in the metal hydroxide solution to the perfluorosulfonamide is (1-1.5): 1; and
a molar ratio of the perfluorosulfonamide metal salt to the organic base is (0.5-1): 1.

10. The preparation method according to claim 6, wherein a temperature for the reacting of the metal hydroxide solution and the perfluorosulfonamide solution is 40-60° C.;
a time for the reacting of the metal hydroxide solution and the perfluorosulfonamide solution is 5-8 h;
a temperature for the mixing the organic base, the polar solvent and the perfluorosulfonamide metal salt is 30-50° C.; and
a time for the mixing the organic base, the polar solvent and the perfluorosulfonamide metal salt is 10-14 h.

11. The preparation method according to claim 6, wherein a method for preparing the metal hydroxide solution comprising: providing an alkali solution and a metal salt solution, mixing and reacting to obtain the metal hydroxide solution.

12. The preparation method according to claim 11, wherein the alkali solution comprises an alkali and a second solvent; and/or
the metal salt solution comprises a metal salt and a third solvent.

13. The preparation method according to claim 12, wherein the alkali is selected from one or more of sodium hydroxide, lithium hydroxide, potassium hydroxide;
the metal salt is selected from one or more of zinc salt, titanium salt, tin salt, tantalum salt, aluminum salt, cesium salt, lithium salt;
the second solvent is selected from ethanol, ethylene glycol, butanol, glycerol, trimethoxybutanol, and methanol; and
the third solvent is selected from one or more of N,N-dimethylformamide, dimethyl sulfoxide, ethylene glycol monomethyl ether, ethylene glycol monoethyl ether, and ethylene glycol monobutyl ether.

14. The preparation method according to claim 12, wherein a mixed time for the alkali solution and the metal salt solution is 2-8 h; and
a molar ratio of the alkali to the metal salt is (1.1-1.3):1.

15. A photoelectric device, comprising:
an anode;
a cathode;
an emission layer, located between the anode and the cathode; and
an electron functional layer, located between the emission layer and the cathode;
wherein a material of the electron functional layer comprises composite material, and the composite material comprising:
an inorganic particle; and
a perfluorosulfonamide; wherein the inorganic particle is connected to the perfluorosulfonamide by a chemical bond, and a chemical formula of the perfluorosulfonamide is R—SO₂—NH₂, wherein R is selected from one or more of a perfluoroalkyl group, a perfluorocycloalkyl group, a perfluoroheterocycloalkyl group, and a perfluoroamine group.

16. The photoelectric device according to claim 15, wherein a mole percentage of the perfluorosulfonamide in the composite material, is 15-25%;
the inorganic particle comprises one or more of oxygen and fluorine; and/or
a material of the inorganic particle comprises doped metal compound particle or non-doped metal compound particle.

17. The photoelectric device according to claim 15, wherein the perfluoroalkyl group is a linear or branched perfluoroalkyl group with 1 to 10 carbon atoms in the main chain, a linear or branched perfluoroalkenyl group with 1 to 10 carbon atoms in the main chain, a linear or branched perfluoroalkynyl group with 1 to 10 carbon atoms in the main chain, or a combination of these groups;
the perfluorocycloalkyl group has 1 to 10 ring atoms;
the perfluoroheterocycloalkyl group has 1 to 10 ring atoms; and
the perfluoroamine group has 1 to 10 carbon atoms in the main chain.

18. The photoelectric device according to claim 16, wherein the perfluorosulfonamide is selected from one or more of perfluorooctylsulfonamide, N-ethyl perfluoro-n-octanesulfonamide, perfluoroethanesulfonamide, perfluorocyclohexanesulfonamide, perfluorocyclopentanesulfonamide, and perfluorobutanesulfonamide;
a material of the non-doped metal compound particle is selected from one or more of ZnO, TiO₂, SnO, Ta₂O₃, CsF, LiF, CsCO₃, SnO₂, and ZrO₂;
a material of a metal compound particle in the doped metal compound particle is selected from one or more of ZnO, TiO₂, SnO, Ta₂O₃, CsF, LiF, CsCO₃, SnO₂, and ZrO₂, and a doping element in the doped metal compound particle is selected from one or more of aluminum, magnesium, lithium, manganese, yttrium, lanthanum, copper, nickel, zirconium, and cerium;
an average particle size of the inorganic particle is 5-10 nm; and
a thickness of the electron functional layer is 15-30 nm.

19. The photoelectric device according to claim 15, wherein the photoelectric device further comprises a hole functional layer, located between the anode and the emission layer, and the hole functional layer comprises one or more of a hole transport layer and a hole injection layer.

20. The photoelectric device according to claim 19, wherein a material of the anode and the cathode is independently selected from one or more of a metal, a carbon material, and a metal oxide; the metal includes one or more of Al, Ag, Cu, Mo, Au, Ba, Ca, Yb, and Mg; the carbon material includes one or more of graphite, carbon nanotube, graphene, and carbon fiber; the metal oxide includes a doped or undoped metal oxide, including one or more of ITO, FTO, ATO, AZO, GZO, IZO, MZO, and AMO, or a composite electrode including a metal sandwiched between doped or undoped transparent metal oxides, including one or more of AZO/Ag/AZO, AZO/AI/AZO, ITO/Ag/ITO, ITO/AI/ITO, ZnO/Ag/ZnO, ZnO/Al/ZnO, ZnS/Ag/ZnS, ZnS/Al/ZnS, TiO₂/Ag/TiO₂, and TiO₂/Al/TiO₂; and/or
a material of the emission layer comprises one or more of 4,4′-Bis(N-carbazole)-1,1′-biphenyl: tris[2-(p-tolyl)pyridine iridium (III)], 4,4′,4″-tris(carbazole-9-yl)triphenylamine: tris[2-(p-tolyl)pyridine iridium], diaryl anthracene derivatives, stilbene aromatic derivatives, pyrene derivatives, fluorene derivatives, TBPe fluorescent materials, TTPX fluorescent materials, TBRb fluorescent materials, DBP fluorescent materials, delayed fluorescent materials, TTA materials, TADF materials, B—N covalent bond containing polymers, HLCT materials, Exciplex light-emitting materials, CdS, CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, CdZnSeS, CdZnSeTe, CdZnSTe, CdHgSeS, CdHgSeTe, CdHgSTe, HgZnSeS, HgZnSeTe, HgZnSTe, SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, SnPbSTe, GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, CuInS₂, CuInSe₂, AgInS₂, CdSe/CdSeS/CdS, InP/ZnSeS/ZnS, CdZnSe/ZnSe/ZnS, CdSeS/ZnSeS/ZnS, CdSe/ZnS, CdSe/ZnSe/ZnS, ZnSe/ZnS, ZnSeTe/ZnS, CdSe/CdZnSeS/ZnS, InP/ZnSe/ZnS, AMX₃, and BMX₃; wherein A is a Cs⁺ ion, M is a divalent metal cation selected from one or more of Pb²⁺, Sn²⁺, Cu²⁺, Ni²⁺, Cd²⁺, Cr²⁺, Mn²⁺, Co²⁺, Fe²⁺, Ge²⁺, Yb²⁺, Eu²⁺, X is a halide anion selected from one or more of Cl⁻, Br⁻, I⁻; B is an organic amine cation selected from CH₃(CH₂)ₙ₋₂NH³⁺ or [NH₃(CH₂)ₙNH₃]²⁺ with n>2; and/or
a material of the hole injection layer and the hole transport layer is each independently selected from one or more of 4,4′-N,N′-dicarbazolyl-biphenyl, N,N′-diphenyl-N,N′-bis(1-naphthyl)-1,1′-biphenyl-4,4″-diamine, N,N′-diphenyl-N,N′-bis(3-methylphenyl)-(1,1′-biphenyl)-4,4′-diamine, N,N′-bis(3-methylphenyl)-N,N′-bis(phenyl)-spiro, N,N′-di(4-(N,N′-diphenyl-amino)phenyl)-N,N′-diphenylbenzidine, 4,4′,4′-tris(N-carbazolyl)-triphenylamine, 4,4′,4′-tris(9H-carbazol-9-yl)triphenylamine, trichloroisocyanuric acid, terbium-doped phosphate-based green light emitting material, 2,3,6,7,10,11-hexacyano-1,4,5,8,9,12-hexaazatriphenylene, 4,4′,4′-tris(N-3-methylphenyl-N-phenylamino)triphenylamine, poly[(9,9′-dioctylfluorene-2,7-diyl)-co-(4,4′-(N-(4-sec-butyphenyl)diphenylamine))], poly(4-butylphenyl-diphenylamine), poly[bis(4-phenyl) (4-butylphenyl)amine], polyaniline, polypyrrole, poly(p-phenylenevinylene), polyphenylenevinylene, poly[2-methoxy-5-(2-ethylhexyloxy)-1,4-phenylenevinylene], poly[2-methoxy-5-(3′,7′-dimethyloctyloxy)-1,4-phenylenevinylene], copper phthalocyanine, aromatic tertiary amines, polynuclear aromatic tertiary amines, 4,4′-bis(carbazol-9-yl)-1,1′-biphenyl compounds, N,N,N′,N′-tetraarylbenzidines, PEDOT, PEDOT:PSS and derivatives thereof, PEDOT:PSS derivatives doped with s-MoO₃, poly(N-vinylcarbazole) and derivatives thereof, polymethacrylates and derivatives thereof, poly(9,9-octylfluorene) and derivatives thereof, poly(spirofluorene) and derivatives thereof, N,N′-di(naphthalen-1-yl)-N,N′-diphenylbenzidine, spiro-NPB, nanopolycrystalline diamond, microcrystalline cellulose, and tetracyanoquinodimethane, doped graphene, undoped graphene, transition metal oxides, transition metal sulfides, transition metal stannides.
